(19) United States
(12) Patent Application Publication
(10) Pub. No.: US 2008/0292637 A1
(43) Pub. Date: Nov. 27, 2008

(54) Therapeutic Uses of A3 Adenosine Receptor Antibodies

(76) Inventor: Pnina Fishman, Herzliya (IL)

Correspondence Address: NATH & ASSOCIATES, 112 South West Street, Alexandria, VA, 22314, US

(21) Appl. No.: 11/632,898

(22) PCT Filed: Nov. 29, 2006
(86) PCT No.: PCT/IL2006/001375

§ 371 Date: Jan. 19, 2007

Related U.S. Application Data
(60) Provisional application No. 60/740,630, filed on Nov. 30, 2005.

(51) Int. Cl. A61K 39/395; A61P 35/00; A61P 19/10; A61P 19/02

(52) U.S. Cl. 424/141.1; 424/130.1

(57) ABSTRACT

The present invention concerns the use of an anti-A3 adenosine receptor (A3AR) immunoglobulin-based molecule for the preparation of a pharmaceutical composition for the treatment of a pathological condition associated with a high level of expression of A3 adenosine receptor. Further provided by the invention is a method of treating a pathological condition associated with a high level of expression of A3 adenosine receptor in a subject, the method comprising administering to said subject an amount of the anti-A3AR immunoglobulin-based molecule, the amount being effective to treat or prevent said pathological condition. Finally, the present invention provides a pharmaceutical composition for the treatment of a pathological condition associated with a high level of expression of A3AR comprising a physiologically acceptable carrier and the anti A3AR immunoglobulin-based molecule.

### FIELD OF THE INVENTION

This invention relates to the use of anti-A₃ adenosine receptor immunoglobulin based molecules in therapy.

### PRIOR ART

The following is a list of prior art which are considered to be pertinent for describing the state of the art in the field of the invention. Acknowledgement of these references herein will at times be made by indicating their number(s) from the list below within parentheses.
1. Fishman, P., Madi, L., Bar-Yehuda, S., Barer, F., Del Valle, L., Khalili, K. Evidence for involvement of Wnt signaling pathway in IB-MECA mediated suppression of melanoma cells. Oncogene, 21:4060-4064 (2002).
2. Fishman, P., Bar-Yehuda, S., Rath-Wolfson, L., Ardon, E., Barer, F., Ochaion, A., Madi, L. Targeting the A₃ adenosine receptor for cancer therapy: inhibition of Prostate carcinoma cell growth by A₃AR agonist. Anticancer Res., 23:2077-2083 (2003).
3. Madi, L., Bar-Yehuda, S., Barer, F., Ardon, E., Ochaion, A., Fishman, P. A₃ adenosine receptor activation in melanoma cells: association between receptor fate and tumor growth inhibition. J. Bio. Chem., 278:42121-42130 (2003).

4. Ohana, G., Bar-Yehuda, S., Arich, A., Madi, L., Dreznick, Z., Silberman, D., Slosman, G., Volfsson-Rath, L., Fishman, P. Inhibition of primary colon carcinoma growth and liver metastasis by the A₃ adenosine receptor agonist IB-MECA. British J. Cancer., 89:1552-1558 (2003).
5. Fishman, P., Bar-Yehuda, S., Ohana, G., Ochaion, A., Engelberg, A., Barer, F., Madi, L. An agonist to the A₃ adenosine receptor inhibits colon carcinoma growth in mice via modulation of GSK-3β and NF-κB. Oncogene, 23:2465-2471 (2004).
6. U.S. Patent Application Publication. No. 20040167094 A1.
7. Szabo, C., et al. Suppression of macrophage inflammatory protein (MIP)-1αproduction and collagen-induced arthritis by adenosine receptor agonists. British J. Pharmacology 125:379-387 (1998).
8. Mabley, J., et al. The adenosine A₃ receptor agonist, N⁶-(3-iodobenzyl)-adenosine-5′-N-methyluronamide, is protective in two murine models of colitis. Europ. J. Pharmacology, 466:323-329 (2003).
9. Baharav, E., et al. The effect of adenosine and the A₃ adenosine receptor agonist IB-MECA on joint inflammation and autoimmune diseases models. Inter. J. Mol. Med. 10 (supplement 1) page S104, abstract 499 (2002).
10. Madi, L., Ochaion, A., Rath-Wolfson, L., Bar-Yehuda, S., Erlanger, A., Ohana, G., Harish, A., Merimski, O., Barer, F., Fishman, P. The A₃ Adenosine Receptor is Highly Expressed in Tumor vs. Normal Cells: Potential Target for Tumor Growth Inhibition. Clinical Cancer Research 10: 4472-4479, 2004.
11. Gessi, S. et al. Elevated expression of A₃ adenosine receptors in human colorectal cancer is reflected in peripheral blood cells Clinical Cancer Research 10:5895-5901, 2004
12. U.S. Patent Application Publication. No. 2004/0137477 A1.

### BACKGROUND OF THE INVENTION

The A₃ adenosine receptor (A₃AR), a Gᵢ protein-associated cell surface receptor, has been proposed as a target to combat cancer and inflammation. The receptor is highly expressed in various tumor cell types while low expression was shown in adjacent normal tissues. Activation of the receptor by a specific synthetic agonist induces modulation of downstream signal transduction pathways which include the Wnt and the NF-κB, resulting in tumor growth inhibition (1-5).

A₃AR agonists were also shown to act as anti-inflammatory agents by ameliorating the inflammatory process in different experimental autoimmune models such as rheumatoid arthritis and Crohn's disease (6-9).

A₃AR expression levels are elevated in cancer cells as compared to normal cells (10, 11). Thus, the A₃AR expression level has been proposed as a marker for the diagnosis of cancer (12). In addition, A₃AR expression levels have also been described to be elevated in peripheral blood cells of patients with colorectal cancer (11).

### SUMMARY OF THE INVENTION

The present invention is based on in vitro as well as in vivo experiments showing that an anti-A₃AR antibody was effective in inhibiting tumor growth and to prevent the development of tumor metastasis.

Furthermore, it has now been shown that the anti-A₃AR antibody modulated protein levels involved in the Wnt and NF-κB signal transduction pathways, both of which play an important role in the pathology of diseases which are characterized by hyper-proliferation of cells, such a cancer or autoimmune inflammatory diseases. Among the characterizing features of cancer and inflammatory cells is also the over-expression of the A₃AR (10-11). Thus, according to the invention anti-A₃AR immunoglobulin-based molecules can be used for treating cancer, inflammatory and a variety of other pathological conditions associated with elevated expression of the receptor.

Thus, the present invention provides the use of an anti-A₃AR immunoglobulin-based molecule, in the preparation of a pharmaceutical composition for the treatment or prevention of a pathological condition associated with over-expression of A₃AR.

The invention also provides a pharmaceutical composition comprising as active ingredient an amount of an anti-A₃AR immunoglobulin-based molecule, the amount being effective to treat or prevent a pathological condition associated with over-expression of A₃AR.

Yet further, the invention provides a method for the treatment or prevention of a pathological condition associated with over-expression of A₃AR, the method comprising providing a subject in need an amount of an anti-A₃AR immunoglobulin-based molecule, the amount being effective to treat or prevent said condition.

Pathological conditions treatable in accordance with the invention include, without being limited thereto, autoimmune disorders, such as Rheumatoid Arthritis and Crohn's disease, osteoarthritis, Sjogren's syndrome; psoriasis neurodegenerative disorders, such as Alzheimer's and Multiple Sclerosis; cancer including solid tumors and lymphoma, or conditions associates with dry eye, all of which exhibit an abnormal increase in expression (over-expression) of the A₃AR on pathological cells associated with the pathological condition.

The anti-A₃AR immunoglobulin-based molecule include anti-A₃AR antibodies, immunological fragments thereof, as well as fusion molecules or conjugates comprising said antibodies or fragments, conjugated or fused to another molecular entity as further detailed below.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1C are bar graphs showing the effect of A₃AR antibodies on the proliferation of different tumor cell cultures, including B16-F10, murine melanoma cells (FIG. 1A); BxPC3, human pancreatic carcinoma cells (FIG. 1B); and LnCap, human prostate carcinoma cells (FIG. 1C).

FIG. 2A-2G are Western Blot analyses of protein extracts derived from LnCap, human prostate carcinoma cell cultures incubated in the presence of A₃AR antibody in comparison to control cell cultures which are not treated with the A₃AR antibody; the protein extracts being A₃AR (FIG. 2A), PKB-Akt (FIG. 2B), NF-κB (FIG. 2C), GSK-P (FIG. 2D), GSK-3β (FIG. 2E), C-Myc (FIG. 2F), Cyclin D1 (FIG. 2G).

FIG. 3 is a bar graph showing the effect of A₃AR antibody on the development B16-F10 Melanoma foci in lung of mice inoculated with melanoma cells.

FIGS. 4A-4B are bar graphs showing the inhibitory effect of A₃AR antibody on the growth of human originated (FIG. 4A) or rat originated (FIG. 4B) FLS.

### DETAILED DESCRIPTION OF THE INVENTION

In accordance with the present invention, an anti-A₃AR immunoglobulin-based molecule is used for the treatment of a pathological condition associated with a high level of expression of A₃ adenosine receptor.

As used herein, the term “anti-A₃AR immunoglobulin-based molecules” which at times is used herein interchangeably with the shortened term “A₃AR antibody” means a molecule comprising an immunoglobulin or a fragment thereof, which is capable of binding an A₃AR antigenic determinant. The anti-A₃AR immunoglobulin-based molecules may be monoclonal or polyclonal antibodies, an immunological fragment thereof. The anti-A₃AR immunoglobulin-based molecules may be obtained from an immunized animal, by immortalizing immunoglobulin producing B-cells and harvesting the produced monoclonal antibodies or may be obtained by synthetic or recombinant means. The anti-A₃AR immunoglobulin-based molecules may also at times be a fusion protein between an immunological fragment and a protein, polypeptide or peptide fusion partner. The antibodies of the present invention may exist in many forms including whole bivalent antibody molecules, monovalent Fab fragments, divalent F(ab′)2 and chemically formed chimeric antibodies, as known in the art (Harlow et al. (1999), Using Antibodies: A Laboratory Manual, Cold Spring Harbor Laboratory Press, NY; Harlow et al. (1989), Antibodies: A Laboratory Manual, Cold Spring Harbor, N.Y.; Houston et al. (1988), Proc. Natl. Acad. Sci. USA 85: 5879-5883; Bird et al. (1988), Science 242: 423-426) including immunological fragments of A₃AR antibodies.

As used herein, the term “immunological fragment” refers to a functional fragment of an antibody that is capable of binding an A₃AR antigenic determinant. Suitable immunological fragments may be, for example, a complementarity-determining region (CDR) of an immunoglobulin light chain (“light chain”), a CDR of an immunoglobulin heavy chain (“heavy chain”), a variable region of a light chain, a variable region of a heavy chain, a light chain, a heavy chain, an Fd fragment, and immunological fragments comprising essentially whole variable regions of both light and heavy chains, such as Fv, single-chain Fv, Fab, Fab′, and F(ab′)₂.

The term “antigenic determinant” is used herein to denote a region or regions, on a protein which may induce the production of antibodies which bind specifically to the antigenic determinant. An antigenic determinant may compete with the intact antigen (i.e., the immunogen used to elicit the immune response) for binding to an antibody. Antigenic determinants typically consist of chemically active surface groupings of molecules such as amino acids or carbohydrate side chains, and usually have specific three-dimensional structural characteristics, as well as specific charge characteristics. In the context of the present invention, the term “antigenic determinant” refers specifically to a region or regions on A₃AR (epitope).

The antibody may be a monoclonal antibody (Mab) or a polyclonal antibody. Methods of generating antibodies (monoclonal or polyclonal) are well known in the art, including, without being limited thereto, the induction of in vivo production of antibody molecules, screening of immunoglobulin libraries (Orlandi, D. R. et al. (1989), Proc Natl Acad Sci USA 86, 3833-3837; Winter G. et al. (1991), Nature 349, 293-299), or generation of monoclonal antibody molecules by continuous cell lines in culture. The latter include, without limitation, the hybridoma technique, the human B-cell hybridoma technique, and the Epstein-Barr virus (EBV) hybridoma technique, which are well known in the art (Kohler, G. et al. (1975), Nature 256, 495-497; Kozbor, D. et al. (1985), J Immunol Methods 81, 31-42; Cote, R. J. et al. (1983), Proc Natl Acad Sci USA 80, 2026-2030; and Cole, S. P. et al. (1984), Mol Cell Biol 62, 109-120).

Monoclonal antibodies which provide single epitope specificity are typically produced by cell lines or clones obtained from animals that have been immunized with the substance that is the subject of study. To produce the desired mAb, cells must be grown in either of two ways: by injection into the abdominal cavity of a suitably prepared mouse or by tissue culturing cells in plastic flasks.

Polyclonal antibodies provide multiple specificity and their production procedures are also known to those versed in the art. In general, polyclonal antibodies are produced in vivo in response to immunization with different epitopes on an immunogen. Anti-serum can be raised in a wide range of animals with multiple injections of the antigen along with the adjuvant (a non-specific enhancer of the immune response). For many small molecules or haptens, a carrier protein (which provides determinants recognized by helper T-cells) is required for conjugation via various bi-functional coupling reagents. Upon repeated immunizations, the antibodies produced are predominantly IgG with a reasonable high affinity. A review of procedures for producing polyclonal antibodies is found in, e.g., Hanley, W. C. et al. (1995), Review of Polyclonal Antibody Production Procedures in Mammals and Poultry, ILAR J 37 (3).

Immunological fragments can be obtained using methods well known in the art. (see, e.g., Harlow and Lane, “Antibodies: A Laboratory Manual”, Cold Spring Harbor Laboratory, New York (1988)). For example, immunological fragments can be prepared by proteolytic hydrolysis of the antibody or by expression in E. coli or mammalian cells (e.g., Chinese hamster ovary (CHO) cell culture or other protein expression systems) of DNA encoding the fragment.

Alternatively, immunological fragments can be obtained by pepsin or papain digestion of whole antibodies by conventional methods. (Fab′)₂ immunological fragments can be produced by enzymatic cleavage of antibodies with pepsin to provide a 5S fragment. This fragment can be further cleaved using a thiol reducing agent, and optionally a blocking group for the sulfhydryl groups results from cleavage of disulfide linkages to produce 3.5S Fab′ monovalent fragments. Alternatively, enzymatic cleavage using pepsin produces two monovalent Fab′ fragments and an Fc fragment directly. Other methods of cleaving antibodies, such as separation of heavy chains to form monovalent light-heavy chain fragments, further cleavage of fragments, or other enzymatic, chemical, or genetic techniques, may also be used, so long as the fragments bind to the antigenic determinant that is recognized by the intact antibody.

Further, as the Fv is composed of paired heavy chain variable and light chain variable domains (an association which may be noncovalent (e.g., Inbar et al. (1972), Proc Natl Acad Sci USA 69, 2659-62)), the variable domains can be linked to generate a single-chain Fv by an intermolecular disulfide bond, or alternately, such chains may be cross-linked by chemicals such as glutaraldehyde.

Single-chain Fvs may also be prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single-chain Fvs is provided in the literature of the art (e.g., Whitlow and Filpula (1991), Methods 2, 97-105; Bird et al. (1988), Science 242, 423-426; Pack et al. (1993), Bio/Technology 11, 1271-1277).

Isolated CDR peptides can be obtained by constructing genes encoding the CDR of an antibody of interest. Such genes may be prepared, for example, by RT-PCR of mRNA of an antibody-producing cell. Ample guidance for practicing such methods is provided in the literature of the art (e.g., Larrick and Fry (1991), Methods 2, 106-110).

Antibody fusion proteins or chimeric antibodies may also be used [Antibody Fusion Proteins, Steven M., Chamow (Editor), Avi Ashkenazi (Editor) April 1999], for example, to form humanized antibodies. Humanized forms of non-human (e.g., murine) antibodies may be genetically engineered chimeric antibodies or immunological fragments having (preferably minimal) portions derived from non-human antibodies. “Humanized antibodies” include antibodies in which CDRs of human antibody (recipient antibody) are replaced by residues from CDRs of non-human species (donor antibody), such as mouse, rat, or rabbit, which have the desired functionality. In some instances, Fv framework residues of the human antibody are replaced by corresponding non-human residues. Humanized antibodies may also comprise residues found neither in the recipient antibody nor in the imported CDR or framework sequences. In general, the humanized antibody will comprise at least one, and typically two, variable domains, in which all or substantially all of the CDRs correspond to those of a non-human antibody, and all, or substantially all, of the framework regions correspond to those of a relevant human consensus sequence. Humanized antibodies optimally also include at least a portion of an antibody constant region, such as an Fc region, typically derived from a human antibody (see, e.g., Jones et al. (1986), Nature 321, 522-525; Riechmann et al. (1988), Nature 332, 323-329; and Presta (1992), Curr Op Struct Biol 2, 593-596.) In practice, humanized antibodies may be typically human antibodies in which some complementarity-determining region residues and possibly some framework residues are substituted by residues from analogous sites in rodent antibodies.

Humanized antibodies can also be produced using various techniques known in the art, including phage display libraries (Hoogenboom and Winter (1991), J Mol Biol 227, 381; Marks et al. (1991), J Mol Biol 222, 581; Cole et al. (1985), “Monoclonal Antibodies and Cancer Therapy”, Alan R. Liss, ed., pp. 77; Boerner et al. (1991), J Immunol 147, 86-95). Humanized antibodies can also be made by introducing sequences encoding human immunoglobulin loci into transgenic animals, e.g., into mice in which the endogenous immunoglobulin genes have been partially or completely inactivated. Upon antigenic challenge, human antibody production is observed in such animals, closely resembling that seen in humans in all respects, including gene rearrangement, chain assembly, and antibody repertoire. Ample guidance for practicing such an approach is provided in the literature of the art (e.g., Marks et al. (1992), Bio/Technology 10, 779-783; Lonberg et al. (1994), Nature 368, 856-859; Morrison (1994), Nature 368, 812-13; Fishwild et al. (1996) Nature Biotechnol 14, 845-851; Neuberger (1996), Nature Biotechnol 14, 826; and Lonberg and Huszar (1995), Intern Rev Immunol 13, 65-93).

According to one embodiment, the antibody or immunological fragment of an antibody is an immunoglobulin G (IgG) antibody or fragment.

In accordance with yet another embodiment of the invention the A₃AR antibody is a polyclonal antibody, preferably produced against a synthetic peptide.

The anti-A₃ adenosine receptor (A₃AR) immunoglobulin-based molecule utilized in accordance with the invention are molecules which have binding properties to A₃AR which are similar to the binding properties of known (see below) anti-A₃AR antibodies. The term “binding properties” refers to the binding specificity of the A₃AR immunoglobulin-based molecule to the A₃AR antigenic determinant, being similar to that of known anti-A₃AR antibodies.

The binding properties of A₃AR immunoglobulin-based molecule to an A₃AR antigenic determinant, i.e. the specificity to an epitope, may be determined by a variety of techniques readily available and known to those versed in the art. The most common techniques consist of Enzyme-Linked Immunosorbant Assays (ELISA), radioimmunoassays (RIA, using radioisotopes such as I¹²⁵) or fluorescence-based immunoassays. Other techniques include gel Immunoelectrophoresis (e.g. Western blots) and agglutination.

In accordance with one embodiment, the anti-A₃AR immunoglobulin-based molecule has binding properties (specificity) similar to that of the rabbit polyclonal IgG antibody utilized in the present invention, being the H-80 antibody (sc-13938, Santa Cruz Biotechnology, Santa Cruz, Calif., USA).

In accordance with another embodiment, the anti-A₃AR immunoglobulin-based molecule has binding properties similar to that of A3R31-A (purchased from Alpha Diagnostic International, San Antonio, Tex., USA).

The anti-A₃AR immunoglobulin-based molecules have a therapeutic beneficial effect in treatment or prevention of pathological conditions associated with elevated expression of the receptor.

As used herein, the terms “treat”, “treating” and “treatment” refer to the administering of a therapeutically effective amount of an antibody or immunological fragment thereof as defined herein, which binds to A₃AR in a manner effective to achieve a desired biochemical and preferably therapeutic effect on a pathological state. The desired effect may include, without being limited thereto, inhibition of cell proliferation, such as cancer cells or inflammatory cell (e.g. in case of rheumatoid arthritis), as over-expression of A₃AR was exhibited in such pathological states.

Thus, as an example, when the pathological state is cancer, treatment denotes, inter alia, inhibition or reduction of the growth and proliferation of tumor cells: including arresting growth of the primary tumor, decreasing the rate of cancer related mortality, delaying cancer related mortality, which may result in the reduction of tumor size or total elimination thereof from the individual's body, decreasing the rate of occurrence of metastatic tumors, or decreasing the number of metastatic tumors appearing in an individual.

Further, as an example, when referring to an inflammatory state as the pathological condition to be treated (e.g. inflammation and autoimmune disorders), treatment denotes amelioration of undesired symptoms associated with the inflammatory state, prevention of the manifestation of such symptoms before they occur, slowing down the progression of inflammatory state, slowing down the deterioration of symptoms associated with the inflammatory state, slowing down the irreversible damage caused by the chronic stage of the inflammatory state, lessening the severity or cure the inflammatory state, to improve survival rate or more rapid recovery form such an inflammatory state.

It should be noted that in the context of the present invention the term “treatment” also denotes “prophylactic treatment”, i.e. for prevention of the development of the pathological condition, or prevention of re-occurrence of an acute phase of a pathological condition in a chronically ill individual.

Many pathological conditions, such as cancer and inflammatory states (e.g. inflammation or autoimmune disorders) are associated with high level of expression of the A₃AR on pathological cells.

The term “pathological cell” denotes the cells that exhibit an abnormal behavior or phenotype and that are associated with the pathological condition, such as cancer cells in cancer or inflammatory cells in inflammation.

As used herein, the term “high level” is to be understood as meaning a significantly higher level of expression of the receptor as compared to expression in normal cells. High level may be determined by comparing to a control level, the control level (reference standard) being the level of A₃AR expression in normal cells (e.g. in healthy subjects). At times, it may be useful to determine the expression level by testing an assayed sample from a patient in parallel to one or more reference standards, e.g. one reference standard indicative of a normal state and another indicative of a pathological condition. A scale indicative of normal vs. elevated (i.e. diseased) levels of expression may then be produced in used as a reference in determining a diseased or normal tissue.

Non-limiting examples of pathological conditions which may be treated in accordance with the invention are inflammatory conditions or diseases; neurodegenerative disorders; conditions related to accelerated bone resorption; malignancies; or benign hyperplastic conditions.

The terms “inflammatory state” refers to any condition wherein one of the manifestations is the presentation of inflammation. The inflammation may be the underlying cause of the pathological condition, or may be the result of another physiological process underlying the condition. This term refers to any state of active or sub-clinical inflammation, including immune-induced pathologies (e.g., autoimmune disorders). The inflammation may be due to an inflammatory disease, or it may be a side effect of some other type of disease or disorder. Pathological cells overexpressing A₃AR may be a variety of inflammatory cells such as synoviocytes and cells associated with bone formation in the case joint inflammations (rheumatoid arthritis, osteoarthritis), lymphocytes, neutrophils, dendritic cells and macrophages.

The following are non-limiting examples of autoimmune diseases which may be treated in accordance with the present invention: The following is a non-limiting list of autoimmune diseases which may be treated in accordance with the present invention: Tropical spastic paraparesis, Acute necrotizing hemorrhagic leukoencephalitis, Paraneoplastic, Hashimoto's thyroiditis, Postpartum thyroiditis, Focal thyroiditis, Juvenile thyroiditis, Idiopathic hypothyroidism, Type I (insulin dependent) diabetes mellitus, Addison's disease, Hypophysitis, Autoimmune diabetes insipidus, Hypoparathyroidism, Pemphigus Vulgaris, Pemphigus Foliaceus, Bullous phemphigoid/Pemphigoid gestationis, Cicatrical pemphigoid, Dermatitis herpetiformis, Epidermal bullosa acquisita, Erythema multiforme, Herpes gestatonis, Vitiligo, Chronic urticaria, Discoid lupus, Alopecia universalis/Areata, Psoriasis, Autoimmune hepatitis, Primary biliary cirrhosis, Chronic active hepatitis, Chronic active hepatitis/Primary biliary cirrhosis overlap syndrome, Primary sclerosing cholangitis, Autoimmune hemolytic anemia, Idiopathic thrombocytopenic purpura, Evans syndrome, Heparin-induced thrombocytopenia, Primary autoimmune neutropenia, Autoimmune (primary) neutropenia of infancy, Autoimmune neutropenia following bone marrow transplant, Acquired autoimmune hemophilia, Autoimmune gastritis and pernicious anemia, Coeliac disease, Crohn's disease, Ulcerative colitis, inflammatory bowel diseases (IBD), Sialadenitis, Autoimmune premature ovarian failure, Azoospermia, Hypogonadism, Male infertility associated with sperm autoantibodies, Autoimmune orchitis, Premature ovarian failure, Autoimmune oophoritis, Uveitis, Retinitis, Sympathetic ophthalmia, Birdshot retinochoroidopathy, Vogt-Koyanagi-Harada granulomatous uveitis, Lens-induced uveitis, Autoimmune myocarditis, Congenital heart block (neonatal lupus), Chagas' disease, Adriamycin cardiotoxicity, Dressler's myocarditis syndrome, Bronchial asthma, Interstitial fibrosing lung disease, Rapidly progressive glomerulonephritis, Autoimmune tubulointerstitial nephritis, Systemic lupus erythematosus (SLE), Antiphospholipid syndrome, Rheumatoid arthritis, Juvenile Rheumatoid arthritis, Felty's syndrome, Large granular lymphocytosis (LGL), Sjogren's syndrome, Systemic sclerosis (scleroderma), Crest syndrome, Mixed connective tissue disease, Polymyositis/dermatomyositis, Goodpasture's Disease, Wegener's granulomatosis, Churg-Strauss syndrome, Henoch-Schonlein purpura, Microscopic polyangiatis, Periarteritis nodosa, Bechet's syndrome, Atherosclerosis, Temporal (giant) cell arteritis, Takayasu arteritis, Kawasaki disease, Ankylosing spondilitis, Reiter's disease, Sneddons disease, Autoimmune polyendocrinopathy, candidiasis-ectodermal dystropy, Essential cryoglobulinemic vasculitis, Cutaneous leukocytoclastic angiitis, Lyme disease, Rheumatic fever and heart disease, Eosinophilic fasciitis, Paroxysmal cold hemoglobinuria, Polymyalgia rheumatica, Fibrolmyalgia, POEMS syndrome (polyneuropathy, organomegaly, endocrinopathy, M-spot and skin changes), Relapsing polychondritis, Autoimmune lymphoproliferative syndrome, TINU syndrome (acute tubulointerstitial nephritis and uveitis), Common variable immunodeficiency, TAP (transporter associated with antigen presentation) deficiency, Omenn syndrome, HyperIgM syndrome, BTK agammaglobulinemia, Human immunodeficiency virus and Post bone-marrow-transplant.

According to a preferred embodiment, the inflammatory state treated in accordance with the invention is an autoimmune inflammatory disease selected from rheumatoid arthritis, Crohn's disease & colitis (collectively referred to as IBD-Inflammatory Bowel disease) and diabetes mellitus.

A pathological condition in accordance with the invention may also be a neurodegenerative disorder. The term “neurodegenerative disorder” is used to denote an abnormal deterioration of the nervous system resulting in the dysfunction of the system. It includes group of conditions in which there is gradual, generally relentlessly progressive wasting away of structural elements of the nervous system exhibited by any parameter related decrease in neuronal function, e.g. a reduction in mobility, a reduction in vocalization, decrease in cognitive function (notably learning and memory) abnormal limb-clasping reflex, retinal atrophy inability to succeed in a hang test, an increased level of MMP-2, an increased level of neurofibrillary tangles, increased tau phosphorylation, tau filament formation, abnormal neuronal morphology, lysosomal abnormalities, neuronal degeneration, gliosis and demyelination. A neurodegenerative disorder to be treated according to a preferred embodiment of the invention is multiple sclerosis (MS).

A pathological condition in accordance with the invention may also be a “condition related to accelerated bone resorption”. Such conditions include, but are not limited to, osteoporosis, Paget's disease, peri-prosthetic bone loss, osteoarthritis or osteolysis, and hypercalcemia of malignancy. The most common of these disorders is osteoporosis, which in its most frequent manifestation occurs in postmenopausal women and in different cancerous diseases such as breast and prostate carcinoma. Because the disorders associated with bone resorption and bone loss are chronic conditions, it is believed that appropriate therapy will generally require chronic treatment.

A pathological condition in accordance with the invention may also be a malignancy. The term “malignancy” is used to denote a disease in which the target cells which over-express A₃AR are tumor cells, including solid tumors as well as blood tumors, including lymphoma or leukemia.

The term “solid tumors” refers to carcinomas, sarcomas, adenomas, and cancers of neuronal origin, and in fact to any type of cancer which does not originate from hematopoeitic cells, and in particular concerns: carcinoma, sarcoma, adenoma, hepatocellular carcinoma, hepatocellularcarcinoma, hepatoblastoma, rhabdomyosarcoma, esophageal carcinoma, thyroid carcinoma, ganglioblastoma, fibrosarcoma, myxosarcoma, liposarcoma, cohndrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all of the above, as it has been shown in previous studies [DeVita, Jr. V., Hellman S., Rosenberg A S. CANCER Principle & Practice of Oncology. Vol 1&2 Lippincott-Raven PUBLISHERS, Philadelphia, N.Y. 1997] that increased expression of A₃AR can be found not only in the primary tumor site but also in metastasis thereof (WO2004/038419).

According to a preferred embodiment, the cancer is breast cancer, or prostate carcinoma.

A pathological condition in accordance with the invention may also be a “benign hyperplastic condition”, such as adenoma, benign prostate hyperplasia and others that are also associated with over-expression of A₃AR and accordingly lend themselves for treatment in accordance with the invention.

The A₃AR antibody may be used in combination with a physiologically acceptable carrier or excipients, to form a pharmaceutical composition. The purpose of a “pharmaceutical composition” of the invention is to facilitate administration of the A₃AR antibody or an immunological fragment thereof as an active ingredient to an individual in need.

When employed as pharmaceuticals, the anti-A₃AR immunoglobulin-based molecule is usually administered in the form of pharmaceutical compositions to facilitate administration of the anti-A₃AR immunoglobulin-based molecule to an individual in need. This invention also includes pharmaceutical compositions, which contain as the active ingredient, one or more of the anti-A₃AR immunoglobulin-based molecules defined herein, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient or enclosed within a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules, suppositories, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of anti-A₃AR immunoglobulin-based molecule in the pharmaceutical composition and unit dosage form thereof may be varied or adjusted widely depending upon the particular application, the manner or introduction, the potency of the particular molecule, and the desired concentration. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The anti-A₃AR immunoglobulin-based molecule is effective over a wide dosage range and is generally administered in a pharmaceutically effective amount. It, will be understood, however, that the amount of the molecule actually administered will be, determined by a physician, in the light of the relevant circumstances, including the condition to be treated, the chosen route of administration, the actual molecule administered, the age, weight, and response of the individual patient, the severity of the patient's symptoms, and the like.

Preferably, the anti-A₃AR immunoglobulin-based molecule can be formulated for parenteral administration in a suitable inert carrier, such as a sterile physiological saline solution. The dose administered will be determined by route of administration. Preferred routes of administration include parenteral or intravenous administration. A therapeutically effective dose is a dose effective to produce a significant therapeutic response (e.g. anti-inflammatory, anti-cancer etc. response).

Administration of anti-A₃AR immunoglobulin-based molecule by intravenous formulation is well known in the pharmaceutical industry. An intravenous formulation should possess certain qualities aside from being just a composition in which the therapeutic anti-A₃AR immunoglobulin-based molecule is soluble. For example, the formulation should promote the overall stability of the anti-A₃AR immunoglobulin-based molecule as well as other active ingredient(s) if present, also, the manufacture of the formulation should be cost effective. All of these factors ultimately determine the overall success and usefulness of an intravenous formulation.

Other accessory additives that may be included in pharmaceutical formulations of the anti-A₃AR immunoglobulin-based molecule as follow: solvents: ethanol, glycerol, propylene glycol; stabilizers: EDTA (ethylene diamine tetraacetic acid), citric acid; antimicrobial preservatives: benzyl alcohol, methyl paraben, propyl paraben; buffering agents: citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate; and tonicity modifiers: sodium chloride, mannitol, dextrose.

The presence of a buffer is necessary to maintain the aqueous pH in the range of from about 4 to about 8 and more preferably in a range of from about 4 to about 6. The buffer system is generally a mixture of a weak acid and a soluble salt thereof, e.g., sodium citrate/citric acid; or the monocation or dication salt of a dibasic acid, e.g., potassium hydrogen tartrate; sodium hydrogen tartrate, phosphoric acid/potassium dihydrogen phosphate, and phosphoric acid/disodium hydrogen phosphate.

The amount of buffer system used is dependent on (1) the desired pH; and (2) the amount of drug. Generally, the amount of buffer used is in a 0.5:1 to 50:1 mole ratio of buffer:alendronate (where the moles of buffer are taken as the combined moles of the buffer ingredients, e.g., sodium citrate and citric acid) of formulation to maintain a pH in the range of 4 to 8 and generally, a 1:1 to 10:1 mole ratio of buffer (combined) to drug present is used.

In addition, the presence of an agent, e.g., sodium chloride in an amount of about of 1-8 mg/ml, to adjust the tonicity to the same value of human blood may be required to avoid the swelling or shrinkage of erythrocytes upon administration of the intravenous formulation leading to undesirable side effects such as nausea or diarrhea and possibly to associated blood disorders. In general, the tonicity of the formulation matches that of human blood which is in the range of 282 to 288 mOsm/kg, and in general is 285 mOsm/kg, which is equivalent to the osmotic pressure corresponding to a 0.9% solution of sodium chloride.

The intravenous formulation can be administered by direct intravenous injection, i.v. bolus, or can be administered by infusion by addition to an appropriate infusion solution such as 0.9% sodium chloride injection or other compatible infusion solution.

The anti-A₃AR immunoglobulin-based molecule can be administered in a sustained release form, for example a depot injection, implant preparation, or osmotic pump, which can be formulated in such a manner as to permit a sustained release of the molecule. Implants for sustained release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host. The implant is placed in proximity to the site of protein deposits (e.g., the site of formation of amyloid deposits associated with neurodegenerative disorders), so that the local concentration of molecule is increased at that site relative to the rest of the body.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes. Proper formulation is dependent, inter alia, upon the route of administration chosen.

Pharmaceutical compositions of the present invention may, if desired, be presented in a pack or dispenser device, such as an FDA-approved kit, which may also be labeled for treatment of an indicated condition.

The anti-A₃AR immunoglobulin-based molecules are used to treat subjects having or in disposition of developing a pathological condition. To this end, the subject is administered with an amount (an effective amount) of the A₃AR antibody or immunological fragment thereof, the amount being effect to treat or prevent the development of the pathological condition.

As used herein, the term “subject” denotes a mammalian, more preferably a human.

As used herein, the phrase “having or in disposition of developing” describes a subject who has been determined to have a pathological condition or exhibit symptoms thereof or is in disposition of developing the pathological condition, e.g. due to genetic factors, exposure to harmful substances etc.

As used herein, the term “effective amount” means an amount of the antibody or immunological fragment effective to prevent, alleviate, or ameliorate symptoms of pathological condition or prolong the survival of the subject being treated. Determination of a therapeutically effective amount is well within the capability of those skilled in the art, especially in light of the detailed disclosure provided herein. In general, the amount of the antibody or immunological fragment to be administered will depend upon the subject being treated, the severity of the affliction, the manner of administration, the judgment of the prescribing physician, and other factors. An effective amount is typically determined through appropriate dose-finding clinical studies. The manner of determining an effective dose is within reach of a person versed in the art of clinical development.

Depending on the severity and responsiveness of the condition to be treated, dosing can be of a single or a plurality of administrations, with course of treatment lasting from several days to several weeks, or until cure is effected or diminution of the disease state is achieved. The dosing schedule will typically be determined on the basis of the pharmacokinetic (PK) properties of the anti-A₃AR immunoglobulin-based molecule. The manner of determining such PK is also within reach of a person versed in the art of clinical development. It is known that at times, antibodies may retain in the body for several weeks. Thus, the dosing schedule may be from once or several doses a day, to once in several days, once a week, once in several weeks and even once in several months, depending, inter alia, on the half life (t_{1/2}) of the antibody.

Throughout the description and claims of this specification, the singular forms “a” “an” and “the” include plural references unless the context clearly dictates otherwise. Thus, for example, a reference to “an antibody” is a reference to one or more antibodies and equivalents thereof known to those skilled in the art. Throughout the description and claims of this specification, the plural forms of words include singular references as well, unless the context clearly dictates otherwise. Thus, for example, a reference to “antibodies” is a reference to one antibody or an equivalent thereof known to those skilled in the art.

Yet, throughout the description and claims of this specification, the words “comprise” and “contain” and variations of the words, for example “comprising” and “comprises”, mean “including but not limited to”, and are not intended to (and do not) exclude other moieties, additives, components, integers or steps.

The invention will now be described by way of non-limiting examples.

### DESCRIPTION OF SOME EXEMPLARY EMBODIMENTS

### Example 1

### Activation of A₃ Adenosine Receptor (A₃AR) by an A₃AR Antibody Induces Anti-Tumor Effect

### Materials and Methods

### Cell Cultures:

B16-F10—murine melanoma cells (maintained in RPMI 1640 medium supplemented with 10% fetal bovine serum (FBS), 200 mM glutamine, 100 U/ml penicillin and 100 μg/ml streptomycin in a 37° C., 5% CO₂ incubator);

BxPC3—human pancreatic carcinoma cells (maintained in RPMI 1640 medium supplemented with 4.5% glucose, 10% fetal bovine serum (PBS), 200 mM glutamine, 100 U/ml penicillin and 100 μg/ml streptomycin, 1.5 gm/L sodium bicarbonate, 10 mM Hepes buffer, 1.0 mM sodium pyruvate in a 37° C., 5% CO₂ incubator);

LnCap—human prostate carcinoma cells (maintained in RPMI 1640 medium supplemented with 10% fetal bovine serum (FBS), 200 mM glutamine, 100 U/ml penicillin and 100 μg/ml streptomycin in a 37° C., 5% CO₂ incubator).

All cell cultures were transferred to a freshly prepared medium twice weekly.

A₃AR antibody—two antibodies were used in the following examples: (1) H-80 (SC-13938) (purchased from Santa Cruz Biotechnology, Santa Cruz, Calif., USA), a rabbit polyclonal anti-human antibody, used in the experiments with the LnCap and BXPC3 as well with the human FLS; (2) A3R31-A (purchased from ALPHA DIAGNOSTIC INTERNATIONAL, San Antonio, Tex., USA), a polyclonal Rabbit anti-rat antibody, which was used in the rat FLS and in the B16-F10 in vitro and in vivo studies

### Thymidine Incorporation Assay:

A ³[H]-thymidine incorporation assay was used to evaluate the effect of an A₃AR antibody (H-80, Santa Cruz) on the growth of B16-F10, BxPC3 or LnCap cells. The cells were serum-starved overnight and then 5×10⁴/ml cells were incubated in the presence of A₃AR antibody at various concentrations in 96-well microtiter plates for 24 hours in the growth medium supplemented with 1% FBS. For the last 18 hours of incubation, each well was pulsed with 1 μCi ³[H]-thymidine. Cells were harvested and the ³[H]-thymidine uptake was determined in an LKB liquid scintillation counter (LKB, Piscataway, N.J., USA).

### Western Blot Analysis:

Western blot analysis (WB) of paw extracts were carried out according to the following protocol. Samples were rinsed with ice-cold PBS and transferred to ice-cold lysis buffer (TNN buffer, 50 mM Tris buffer pH=7.5, 150 nM NaCl, NP 40). Cell debris was removed by centrifugation for 10 min, at 7500×g. Protein concentrations were determined using the Bio-Rad protein assay dye reagent. Equal amounts of the sample (50 μg) were separated by SDS-PAGE, using 12% polyacrylamide gels. The resolved proteins were then electroblotted onto nitrocellulose membranes (Schleicher & Schuell, Keene, N.H., USA). Membranes were blocked with 1% BSA and incubated with the desired primary antibody (dilution 1:1000) for 24 h at 4° C. Blots were then washed and incubated with a secondary antibody for 1 h at room temperature. Bands were recorded using BCIP/NBT color development kit (Promega, Madison, Wis., USA). Data presented in the different figures are representative of at least four different experiments.

### Murine Melanoma Lung Metastases Model:

To test the effect of the A₃AR antibody in vivo, the murine melanoma lung metastases model was utilized. B16-F10 melanoma cells (2.5×10⁵ cells in 250 μl PBS) were intravenously injected to the tail vein of male C57BL/6J mice. A₃AR antibody (1 μg/kg) was administered intraperitoneally 24 hours after tumor inoculation. After 14 days the lungs were removed and the lung metastatic foci were counted under a binocular.

### Results

### In Vitro Effect of A₃AR Antibody:

The in vitro effect of A₃AR antibodies on thymidine incorporation was examined in three different cell lines as described above.

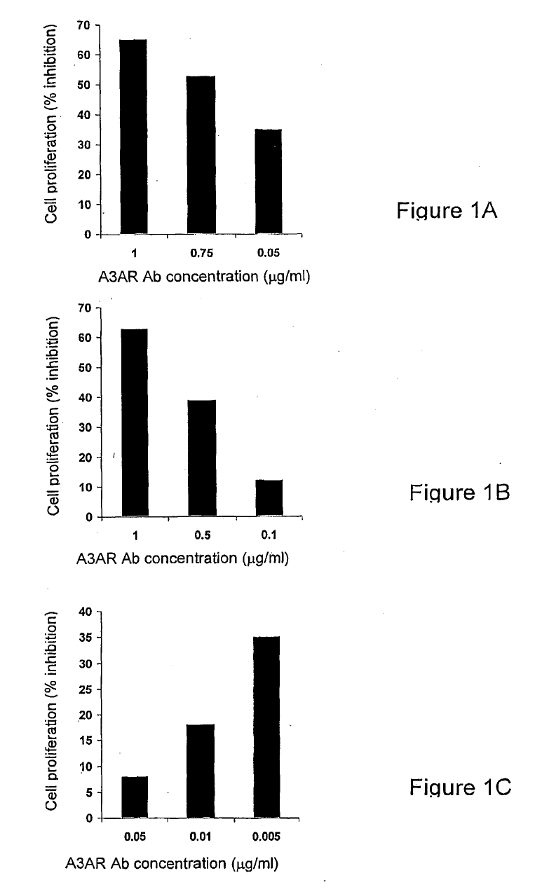

FIGS. 1A-1C show that addition of A₃AR antibody to the growth medium of B16-F10 melanoma cells, BxPC3 pancreatic carcinoma cells or LnCap human prostate carcinoma cells inhibited the proliferation of the cells.

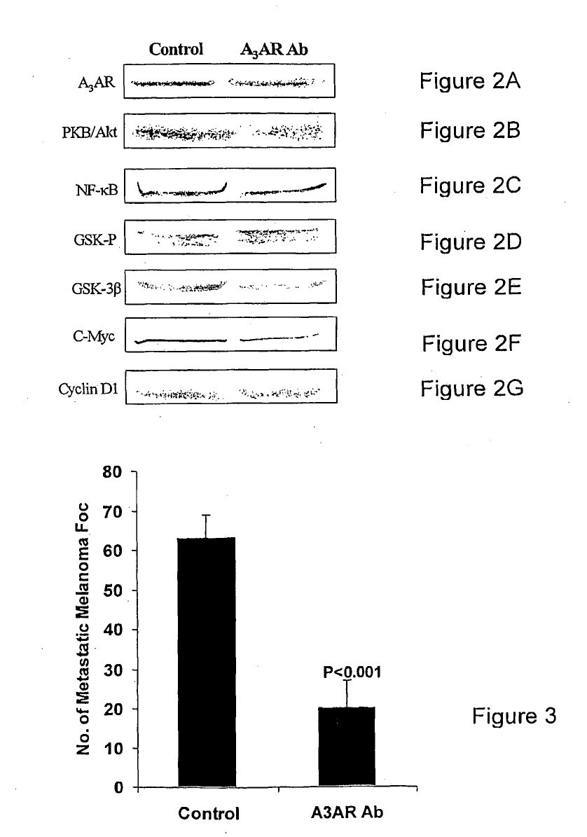

Further, the effect of A₃AR antibodies in the A₃ adenosine receptor's signaling pathway was determined. To this end, protein extract derived from LnCap human prostate carcinoma cells treated with 0.005 μg/ml of A₃AR antibody was examined. The protein profile of LnCap human prostate carcinoma cells is shown in FIG. 2A-2G, which reveals down-regulation in the A₃AR protein expression level as well as modulation in the expression levels of proteins participating in the Wnt and the NF-κB signaling pathways. The levels of PKB/Akt and NF-κB were down-regulated, demonstrating that an inhibition of the NF-κB signaling pathway occurred. On the other hand, modulation in the expression level of GSK-3β (up-regulation of the phosphorylated GSK-3β and down-regulation in the level of the total GSK-3β), which is a key element of the Wnt signaling pathway, demonstrated that inhibition of the latter was induced by A₃AR antibody treatment. As a result of the above events, cell growth inhibition was induced. This is demonstrated by down-regulation in the expression levels of Cyclin D1 and c-Myc, which are proteins playing a pivotal role in cell cycle progression.

### In Vivo Effect of A₃AR Antibody:

To test the effect of the A₃AR antibody in vivo, the murine Melanoma lung metastases model was utilized as described above. FIG. 3 demonstrates the ability of A₃AR antibody administration to inhibit the development of melanoma lung metastasis in vivo.

### Example 2

### Activation of A₃ Adenosine Receptor (A₃AR) by an A₃AR Antibody Inhibits Proliferation of Fibroblasts Like Synoviocytes (FLS)

### Materials and Methods

Fibroblast like synoviocytes (FLS)—FLS was derived from synovia tissue obtained from osteoarthritis patients undergoing paracenthesis or from adjuvant induced arthritis rats. Specifically, dissected synovial tissues were digested by collagenase (4 mg/ml) at 37° C. for 1 hour. The resulting synovial cells were maintained in complete Dulbecco's minimum essential medium supplemented with 10% fetal calf serum, 100 U/ml penicillin and 100 μg/ml streptomycin in a 37° C., 5% CO₂ incubator. The cultured FLS were transferred to a freshly prepared medium twice weekly

³[H]-Thymidine incorporation assay: ³[H]-Thymidine incorporation assay was used to evaluate the effect of an A₃AR antibody on the FLS cultures (H-80, for the human originated FLS and C-31 for the rat originated FLS). FLS at passages 5-8 (5×10⁴/ml cells) were incubated in the presence A₃AR antibody (0.05 and 0.75 μg/ml) in 96-well microtiter plates for 72 hours in the growth medium. For the last 18 h of incubation, each well was pulsed with 1 μCi [³H]-thymidine. Cells were harvested and the [³H]-thymidine uptake was determined in an LKB liquid scintillation counter (LKB, Piscataway, N.J., USA).

### Results

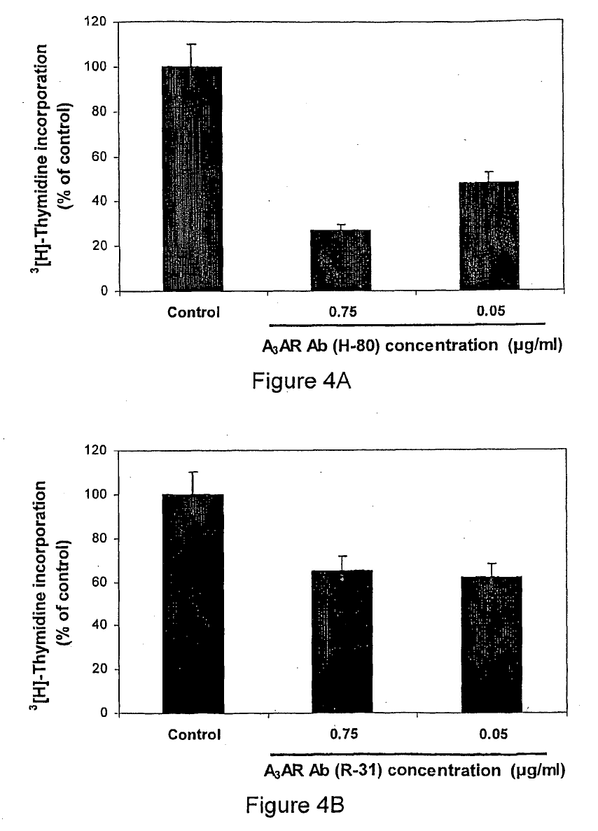

The addition of A₃AR antibody to the growth medium of human originated (FIG. 4A) or rat originated (FIG. 4B) FLS inhibited proliferation of these cells. This result is illustrative of the anti-inflammatory activity of the A₃AR antibody.

1-20. (canceled)

21. A method of treatment of a pathological condition associated with a high level of expression of A₃ adenosine receptor in a subject, the method comprising administering to said subject an amount of an anti-A₃AR immunoglobulin-based molecule, the amount being effective to treat or prevent said pathological condition.

22. The method of claim 21, wherein said anti-A₃AR immunoglobulin-based molecule is a polyclonal antibody.

23. The method of claim 21, wherein said anti-A₃AR immunoglobulin-based molecule is a monoclonal antibody (Mab).

24. The method of claim 21, wherein said anti-A₃AR immunoglobulin-based molecule is specific to the third cytoplasmic loop of A₃AR.

25. The method of claim 21, wherein said anti-A₃AR immunoglobulin-based molecule is produced against a synthetic peptide.

26. The method of claim 21, wherein said anti-A₃AR immunoglobulin-based molecule is an IgG antibody.

27. The method of claim 21, wherein said anti-A₃AR immunoglobulin-based molecule has binding properties similar to an anti-A₃AR antibody selected from ab13161, H-80 or A3R31-A.

28. The method of claim 21, wherein said pathological condition is selected from an inflammatory condition, malignancy, neurodegenerative disorder, a condition related to accelerated bone resorption, a benign hyperplastic disorder, psoriasis and dry eye.

29. The method of claim 28, wherein said inflammatory condition is selected from Rheumatoid Arthritis, Crohn's disease, osteoarthritis, Sjogren's syndrome.

30. The method of claim 28, wherein said neurodegenerative disorder is Multiple Sclerosis.

31. The method of claim 28, wherein said malignancy is breast cancer, colon cancer or melanoma.

32. The method of claim 28, wherein said condition related to accelerated bone resorption is osteoporosis.
